(19) United States
(12) Patent Application Publication
(10) Pub. No.: US 2023/0166133 A1
(43) Pub. Date: Jun. 1, 2023

(54) RESPIRATORY AIR DISINFECTION DEVICE, RESPIRATORY PROTECTION MASK WITH SAME AND RESPIRATORY AIR DISINFECTION METHOD WITH SAME

(71) Applicant: Q One Holding AG, Sarnen (CH)

(72) Inventor: Bernd Hoehne, Achim (DE)

(21) Appl. No.: 17/916,846

(22) PCT Filed: May 12, 2021
(86) PCT No.: PCT/EP2021/062703

§ 371 Date: Oct. 4, 2022

(30) Foreign Application Priority Data
May 13, 2020 (DE) 10 2020 112 999.8

(51) Int. Cl. A62B 18/08; A61L 9/20; A62B 23/02

(52) U.S. Cl. CPC A62B 18/08; A61L 9/20; A62B 23/02

(57) ABSTRACT

A respiratory air disinfection device having a respiratory air channel (1), through which respiratory air is conducted to and/or from a person via an inlet portion (11) in a first direction of flow (X). UVC radiation means (3) are disposed in a widened section (2) of the respiratory air channel (1) to disinfect the respiratory air. The widened section (2) of the respiratory air channel (1) is widened on all sides in all radial directions (R), and a flow surface (22) hindering through-flow of the respiratory air in a straight line is provided in the region with the greatest widening, which surface deflects the respiratory air outwards in all radial directions (R) to the first direction of flow (X) in this widened section (2) of the respiratory air channel (1).

The invention relates to a respiratory air disinfection device with a respiratory air channel through which respiratory air is supplied and/or discharged via an inlet section in a first flow direction of a person, wherein a widened section of the respiratory air channel is provided and in the widened section of the respiratory air channel UVC radiation agents for disinfection of the respiratory air are arranged. Furthermore, the invention relates to a respirator with such respiratory air disinfection devices and a respiratory air disinfection method therewith.

Herein, the first direction of flow of the respiratory air is to be understood as a general flow direction which leads along the longitudinal axis of the respiratory air channel, which is formed, for example, as a breathing tube or the like, to or away from a person.

From US 2016/0001108 A1 a respiratory air disinfection device is known that has UV radiation means in a flow chamber that disinfects the through-flowing air. The respiratory air is passed through a flow chamber and agitated there through structures, so that the air flow path is effectively extended. Alternatively, the flow chamber is divided into several serpentine-like respiratory air channels, which lengthen the airway and thus improve air purification.

The post-published DE 10 2020 106 235 B3 describes a respirator in which respiratory air is passed through a chamber interior in which ultraviolet emitting light sources are arranged. The chamber interior can be formed in two chamber sections by means of a separation device, wherein the chamber sections are fluidically connected.

The object of the invention is therefore to provide a respiratory air disinfection device or method with which disinfection of the respiratory air is possible in a reliable and energy-efficient manner.

This object is achieved with a respiratory air disinfection device according to claim 1 and a respiratory air disinfection method according to claim 8.

By forming a widened section of the respiratory air channel as an all-round widening in all radial directions, wherein in the area of the greatest expansion a surface is provided obstructing the straight flow of the respiratory air, causing a deflection of the respiratory air perpendicular to the first direction of flow in all radial directions outwards in this widened section of the respiratory air channel, and the thus effectively widening the flow cross-section in the widened section compared to the flow cross-section in the inlet section, the flow path of the respiratory air in the device is extended and at the same time the flow cross-section in this widened section of the respiratory duct is enlarged.

It is crucial that the respiratory air, which flows in the original first flow direction (general flow direction), is now deflected in all radial directions, i.e. now fanned out over the entire circumferential direction around the first flow direction, in a wider, namely deflected flow direction as a radial flow direction, whereby the effective flow cross-section is distributed over the entire circumference of the breadth of this area and significantly increases in the radial direction outwards with the increasing radius.

Accordingly, the respiratory air guided over this widened section of the respiratory air channel is slowed down in its flow velocity and the flow path to be covered is increased in this widened section. As a result, the exposure time is extended and the effect of UVC-radiation on the respiratory air flowing along this widened section and the germs possibly contained therein improve considerably. It should be borne in mind that UVC-light is already strongly weakened in intensity by the air, i.e. only has a penetration depth of a few millimeters to 10 - 20 mm. Furthermore, it should be taken into account that any intervening separating layers cause an additional, significant attenuation of the radiation intensity.

It should be borne in mind that an average adult person at rest breathes about half a liter of air as respiratory air with one breath over a period of about 1.5 seconds. This results in a flow velocity of approx. 2 m/s for a conventional respiratory air supply hose, as it is also used in hospitals, for example, with an inner diameter of 15 mm. By widening the flow cross-section in the widened section of the respiratory air channel, a significantly reduced speed of a few cm/s preferably 1 cm/s or even less is achieved in the outer periphery area, depending on the geometric design. For this purpose, the effective flow cross-section in the widened section should be increased by a factor of 10 to 500 compared to the flow cross-section in the inlet section, preferably by a factor of 20 to 100.

If the UVC radiation means are arranged in the widened section in the area of the largest flow cross-section expansion, the UVC irradiation acts in the area of the slowest air flow, which is accompanied by a particularly high effectiveness. In view of the extended flow path, the slowed air flow and the possibility of directly arranging the UVC-radiation agents at the periphery of this widened section of the respiratory air channel, efficient active times of 2 - 3 s of the UVC-B radiation on an air particle flowing past can be achieved. Therein it could be found that exposure times of more than 1 s when using conventional UVC-LEDs with, for example, 0.6 watts of power a very effective germ reduction, namely killing of any bacteria, viruses or the like transported in the air.

With regard to the method, this object is performed with the following steps: Redirecting the respiratory air outwards in all radial directions relative to the first direction of flow in a widened section of the respiratory air channel, thereby increasing the flow path and the flow cross-section in this widened section of the respiratory air channel, and irradiating the respiratory air in this widened section of the channel with UVC radiation.

The fact that the respiratory air is exposed to UVC radiation at the radial-outer edge in the area of the greatest expansion of the flow cross-section results in disinfection very effectively in this area of the lowest flow velocity of the air.

If the respiratory air at the radial-outer edge in this widened section of the respiratory air channel is redirected back into the general flow direction, the respiratory air can continue to be led to a breathing mask via appropriately connected tubes, again with the significantly higher air flow velocity forming therein.

Alternatively, for example, two respiratory air disinfection devices can be directly arranged in a respirator mask, wherein then a redirection back to the general direction of the respiratory air can be dispensed with and the so disinfected respiratory air can be directed directly into the interior of the breathing mask.

Furthermore, the UVC-irradiation can be modulated and/or summed to achieve a better penetration depth of UVC-irradiation in the n respiratory air to be disinfected. This is intended to counteract the damping of UVC irradiation in the air we breathe in order to ensure a reinforcing effect of UVC irradiation. Here, a technique already known from radio operation or in the modulation of infrared light or laser light can be used. Both amplitude and frequency modulation are possible. Further, summed signals with UVC-amplifying effect can be generated by adding one or more additional light frequencies.

According to the device, it is preferred in further refinement that the omnidirectional expansion has the shape of a cylinder, wherein the cylinder axis of the cylinder coincides with the first flow direction and the flow surface is a circular disc that is centered and orthogonal to the first flow direction in the cylinder and is arranged in such a way that an annular space between the cylinder jacket surface and the circular disc remains free for further flow deflection. This further flow deflection takes place in the area of the annular chamber near the outer edge of the circular disc, so that here the respiratory air - in an even lower flow velocity - already flows off in the direction of the first flow direction. Depending upon the respective design of the respiratory air disinfection device and the further flow path of the respiratory air, the respiratory air can be brought together again in a narrower single respiratory air tube as a continuous respiratory air channel, whereby the flow velocity of the respiratory air increases again, or the respiratory air is deflected directly or radially slightly inwards at this point of the annular space between the cylinder jacket surface and the circular disc, for example into a breathing mask.

The formation of the all-sided expansion in the form of a cylinder is very easy to implement in terms of production technology and allows a flow-technical distribution of the air flow in this widened section of the breathing duct. Accordingly, there should be an essentially uniform reduction in the flow speed and distribution of respiratory air in this cylinder. According to the extended flow path, which is also realized, a significantly increased exposure time of the UVC-irradiation to the passing respiratory air can be achieved.

To support a uniform ventilation distribution, baffle surfaces are provided in the respiratory air channel and in particular the widened section of the respiratory air channel, which divides the flow of the respiratory air into equivalent flow paths.

If the widened section of the respiratory air channel is formed from a UVC light-transmitting material, for example crystal glass or graphene, in particular plastic, preferably PMME, on which UVC LEDs are arranged as UVC radiation agents, the UVC-LEDs can also be arranged outside the housing. Alternatively, however, the UVC-radiation means in the form of UVC-LEDs can be arranged within the respiratory air channel in the widened section, since in this case a direct irradiation of the respiratory air flowing past is possible without having to take into account the intensity of the UVC-irradiation due to interface transitions that weaken the intensity.

If a liquid separator and/or a particle filter is arranged in the respiratory air channel in the direction of flow of the respiratory air in front of the widened section, undesirable components, namely on the one hand excessive moisture and on the other hand any dirt/dust particles can be filtered out of the respiratory air. This is advantageous for hygienic reasons, since bacteria/viruses are often attached to airborne droplets and/or particles and contamination of the inside of the respiratory disinfection device must be avoided.

If an ultrasonic transducer is arranged on the widened section, cleaning cycles for the air disinfection device can be carried out by controlling the ultrasonic transducer, in which any dirt/dust particles within the air disinfection device, in particular in the widened section, can be cleaned. This can be done in separate service rooms or during normal operation from time to time.

In the following, two illustrative embodiments are described in detail on the basis of the accompanying drawings.

There is shown in:

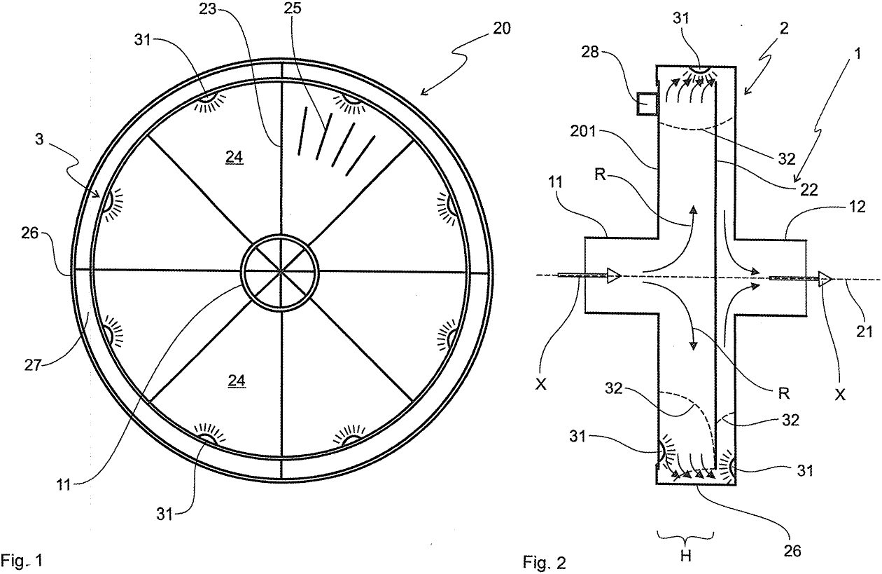

FIG. 1 a respiratory air disinfection device in a first embodiment in a partially sectional view,

FIG. 2 the respiratory air disinfection system in cross-section shown in FIG. 1,

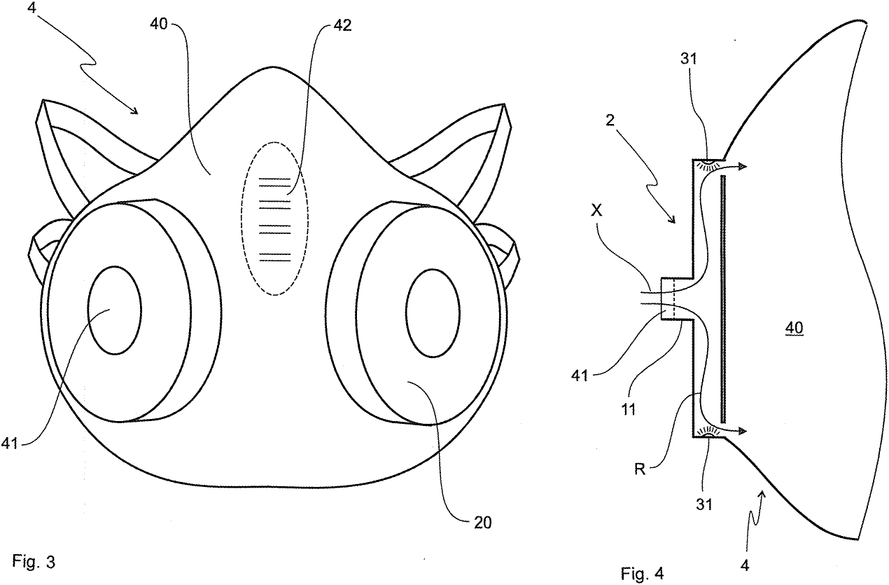

FIG. 3 a second embodiment of the invention in the form of a protective mask with two respiratory air distortion devices in spatial view and

FIG. 4 the respirator shown in FIG. 3 in partially sectional side view.

FIG. 1 shows a respiratory air disinfection device in a first embodiment in a partially sectional view. FIG. 2 shows this respiratory air disinfection device in cross-section. There is shown in FIG. 2 a respiratory air channel 1 with an inlet section 11 with a small diameter, a widened section 2 with a significantly larger diameter (see FIG. 1) and an outlet section 12 in turn with a small diameter.

The widened section 2 of the respiratory air channel 1 is cylindrical or can shaped in the execution example shown here, i.e. has a relatively low cylinder height H and a relatively large cylinder diameter. Arrows with corresponding flow directions are shown to illustrate the flow path for the respiratory air channel 1. The widened section or the all-sided expansion 2, here in cylindrical form, has a cylinder axis 21 which coincides with the first flow direction X, wherein in the widened section 2 in cylindrical form a flow surface in the form of a circular disc 22 is arranged. The widened section 2 is formed from a cylindrical housing 20. In the inlet section 11, the respiratory air flows with a first flow direction X parallel to the longitudinal extension of the respiratory air channel 1 (along the cylinder axis 21 of the corresponding pipe sections) and then divides into second flow direction R, radial to the first flow direction X wherein the respiratory air flow is distributed into the omnidirectional expansion in the cylindrical form section 2, as indicated by the flow arrows marked there with R.

In the partially sectional view according to FIG. 1 it can be seen that in the widened section 2 in cylindrical form baffle surfaces 23 are arranged in such a way that the expanding cavity within the cylinder is divided into eight equal sectors 24. The pie shaped sectors 24 can be divided in the outer periphery by 23 additional air channel walls 25 in addition to the air channels.

In FIG. 1, in the partially sectional view, the circular disc 22 is shown in top view. Between the outer circular edge of the circular disc 22 and the circumferential cylinder jacket surface 26 of the housing 20, an annular space 27 is formed, through which the respiratory air flows around the circular disc 22, and as can be seen in FIG. 2, back to the cylinder axis 21 in cylinder 2 and is redirected back to the first flow direction X subsequently to outlet section 12 of the respiratory air channel 1.

Furthermore, in FIG. 1 on the cylinder jacket surface 26, UVC-radiation means 3 in the form of UVC-LEDs 31 are shown on the inside. In the illustrative examples shown here, a UVC-LED 31 is provided for each sector 24, as shown in FIG. 2 above. Optionally, in the housing 20 of the widened section 2, further UVC-LEDs 31 may be arranged, in particular in the peripheral area, i.e. close to the outer edge of the cylindrical, all-sided expansion 2, as this alternative is shown in FIG. 2, bottom.

In the following, the flow path representation according to FIG. 2 is discussed again. The respiratory air flowing in via inlet section 11 in the flow direction of the first flow direction X is forcibly diverted in the radial flow direction by the circular disc 22 arranged in the widened section 2 in all radial directions R to the first flow direction X. Accordingly, the respiratory air is distributed in fan-like expanding flows in the eight sectors 24 over the entire circumference, whereby by this fan-like expansion over the entire circumference a cross-section enlargement compared to the diameter of the inlet section 11 and at a corresponding effective height H of the cylinder 2 overall, there is a significant slowdown in the flow velocity, as shown by the shorter flow arrows at the peripheral edge.

Precisely at this point (at the peripheral or outer edge of cylinder 2) then preferably also the UVC-radiation means 3 in the form of individual UVC-LEDs 31 are arranged. For example, the UVC-LEDs 31 can be arranged on the inside of the cylinder wall surface 26 in order to be able to radiate directly onto the respiratory air flowing into it. Optionally, supplementary UVC-LEDs 31 are provided on the housing 20 of the cylinder 2, in particular in the cylinder cover 201 of the housing 20 in turn close to the outer peripheral area. As UVC-LED 31, for example, commercially available 0.8 watt LEDs can be used. This type UVC-LEDs 31 have a light intensity in the UVC-range which is suitable for killing microorganisms, in particular bacteria, viruses or the like, wherein the penetration depth of this UVC-radiation into the airspace should be at least 10 mm, preferably at least 20 mm. Accordingly, FIG. 2 shows an effective space 32 with a dash-dot line, in which the UVC radiation emitted by the UVC-LED 31 has a germicidal effect.

Due to the prominent cross-sectional expansion in the area of the widened section or cylinder 2, the flow speed decreases according to this cross-sectional enlargement. Due to the extended flow path in this widened section 2 and the optimal coupling of the UVC-radiation by the UVC-means 3 in the vicinity of the lowest possible flow speed of the respiratory air, namely close to an outer edge of the cylinder 2, the UVC-radiation acts with sufficient germicidal effect over a range of 20 to 40 mm, and when using two UVC-LEDs 31 per sector 24 probably even over up to 60 mm, so that the slow-flowing respiratory air is disinfected with high efficacy due to the germicidal property of UVC radiation. Therein active time per passing air particles of at least one second, most likely for several seconds, are acheivable. Accordingly, the germ number is drastically reduced, which makes the respiratory air in the area of the outlet section 12 practically germ-free.

The widening (enlargement) of the flow cross-section in the airway (respiratory air channel 1) can be calculated in the embodiment shown in FIGS. 1 and 2 from the cylinder circumference multiplied by the effective cylinder height in relation to the cross-section of the supply hose (inlet section 11), for example with
rₛ = radius inlet section 11, e.g. 7.5 mm,
r_{Z} = 5 ^{∗} rₛ = radius cylinder 2,
h_{Z} = 2 ^{∗} rₛ = effective cylinder height, i.e. distance between cylinder cover 201 and circular disc 22,
v₁ = 2 m/s = flow velocity in inlet section 11 and
v₂ = resulting flow velocity near the outer edge of the cylinder
results in:

$$\begin{matrix}π ∗ {\text{r}}_{s}^{2} {\text{*v}}_{1} = 2 * π * {\text{r}}_{\text{Z}} ∗ {\text{h}}_{\text{Z}} * {\text{v}}_{2} \\ {\text{r}}_{\text{s}}^{2} * {\text{v}}_{1} = 2 * 5 * {\text{r}}_{\text{s}} * 2 * {\text{r}}_{\text{s}} * {\text{v}}_{2}\end{matrix}$$

thus:

$${\text{v}}_{2} = 1 / 20 * {\text{v}}_{1} ,$$

whereby the flow velocity, which is 2 m/s at the inlet section, for example, is reduced near the outer edge of the cylinder to 1/20 of the flow velocity, i.e. 0.1 m/s. And it is precisely at this point that the UVC LEDs are to be arranged for an optimal effect, as illustrated by the dash-dot line active space 32 in FIG. 2. The air flows here at a relatively slow speed of 10 cm/s over a range of action of several cm, so that the there-along flowing air is exposed to intensive UVC irradiation for about 0.5 s.

In a second embodiment according to FIGS. 3 and 4, a respirator 4 is equipped with two respiratory air disinfection systems, such as those described in FIGS. 1 and 2. In FIG. 3, the respiratory protective mask 4 is reproduced in spatial view. From FIG. 4, in which a partially sectional partial view of the respirator 4 according to FIG. 3 is shown, the flow path of the respiratory air through the respective air disinfection device is shown. In this second embodiment, components functionally identical with the first illustrative example are given the same reference numbers.xx

In contrast to the first illustrative example, the respiratory air is freely introduced into the protective mask 4 immediately after passing the circular disc 22. Since the respiratory air is advantageously introduced with low flow speed into the internal space of the respirator 4, so that hardly any unpleasant airflow movements are noticeable on the facial skin and hardly any turbulence within the respirator 4 occurs. The respirator 4 has an airtight mask body 40, on which the two air disinfection devices according to FIGS. 1 and 2 are inserted. In this embodiment a fine dust filter 41 is arranged on the free end of the inlet section 11 in each case. Furthermore, an exhalation valve 42 in the form of a check valve is accommodated in the air-tight mask body 40, so that the air from the person can be released directly into the ambient environment after exhalation via this exhalation valve 42. Optionally, a sensor system may be provided in both respiratory air disinfection devices for monitoring the distribution of the air flow.

Of course, alternatively, the respiratory air can also be passed through the air disinfection devices in order to also remove the exhaled air released to the environment from any infected person.

As a further option, an ultra-sound transducer 28 may be provided on the respiratory air disinfection device, in particular on the widened section/cylinder 2, which, when activated, imparts vibration movements generated by the ultrasonic transducer 28 to dust/dirt particles in the area of the respiratory air channel 1, in particular at the widening of the section/cylinder 2. This cleaning can be carried out at specified intervals or in a service mode in order to ensure an optimal effect of UVC-irradiation and at the same time hygienic inner surfaces in the respiratory air disinfection device.

Another way to increase the efficiency of UVC irradiation is to change UVC light by modulating the amplitude or frequency and/or to superimpose the UVC light with one or more light sources with different frequencies in order to obtain a sum light signal in the UVC range, which imparts a higher effect and, if necessary, lower attenuation in the air and, in certain cases, in the UVC-translucent material of cylinder 2.

UVC light modulation with a carrier frequency promotes a transmitting effect through the air. This UVC irradiation leads to demodulation when impinging on special crystalline surfaces/structures and can unfold its UVC curvature there again. These rigid structures for demodulation can be arranged in the housing 20 of cylinder 2 in order to favor the disinfection effect there and to counteract the damping by the air and, if necessary, in the UVC-translucent material of cylinder 2.

Reference number list

| | |
|---|---|
| 1 | respiratory air channel |
| 11 | inlet section |
| 12 | outlet section |
| 2 | widened section, all-round expansion, cylinder |
| 20 | housing |
| 201 | cylinder cover |
| 21 | cylinder axis |
| 22 | flow surface, circular disc |
| 23 | flow control surface |
| 24 | sector |
| 25 | air baffle |
| 26 | cylinder surface |
| 27 | annulus |
| 28 | ultrasonic transducer |
| 3 | UVC-radiation means |
| 31 | UVC-LED |
| 32 | effective space |
| 4 | respiratory protection mask |
| 40 | airtight mask body |
| 41 | fine dust filters, particulate filters |
| 42 | exhalation valve |
| H | height of the cylinder |
| R | radial flow direction |
| X | first flow direction |

1. A respiratory air disinfection device having a respiratory air channel (1), adapted to conduct respiratory air to and/or from a person via an inlet portion (11) in a first direction of flow (X), including a widened section (2) of the breathing air channel (1) and UVC radiation means (3) for disinfecting the breathing air are arranged in the widened section (2) of the breathing air channel (1), wherein the widened section (2) of the breathing air channel (1) widens omnidirectionally in all radial directions (R), wherein in the area of the greatest widening a flow surface (22) is provided which prevents the breathing air from flowing through in a straight line and which causes deflection of the breathing air in all radial directions (R) to the first flow direction (X) outwards in this widened section (2) of the breathing air duct (1), and wherein the effective flow cross section in the widened section (2) is widened compared to the flow cross section in the inlet section.

2. The respiratory air disinfection device according to claim 1, wherein the omnidirectional expansion has the shape of a cylinder (2), wherein the cylinder axis (21) of the cylinder (2) coincides with the first flow direction (X) and the axis of the inlet section (11), and the flow surface is a circular disc (22) which is arranged centrally and orthogonally to the first flow direction (X) in cylinder (2) in such a way that an annular space (27) remains free at the outer edge between cylinder housing surface (26) and circular disc (22) for further flow deflection.

3. The respiratory air disinfection device according to claim 1, wherein the effective flow cross-section in the widened section (2), compared to the flow cross-section in the inlet section (11), is enlarged by a factor of 10 to 500.

4. The respiratory air disinfection device according to claim 3, wherein the effective flow cross-section in the widened section (2), compared to the flow cross-section in the inlet section (11), is increased by a factor of 20 to 100.

5. The respiratory air disinfection device according to claim 1, wherein the UVC radiation means (3) are arranged in the widened section (2) in an area of the largest flow cross-section expansion.

6. The respiratory air disinfection device according to claim 1, wherein baffle surfaces (23) are provided in the widened section (2) of the respiratory air channel (1), which divide the flow of the respiratory air into equal flow paths.

7. The respiratory air disinfection device according to claim 1, wherein the widened section (2) of the respiratory air channel (1) is formed from a UVC light-transmitting material on which UVC LEDs (31) are arranged as UVC radiating means (3).

8. The respiratory air disinfection device according to-any claim 1, wherein a liquid separator and/or a particle filter (41) is arranged in the respiratory air channel (1) in front of the widened section (2) in the direction of flow of the respiratory air.

9. The respiratory air disinfection device according to claim 1, wherein an ultrasonic transducer (28) is arranged on the widened section (2).

10. A respiratory protection mask (4) with at least one respiratory air disinfection device according to claim 1.

11. A respiratory air disinfection method by which respiratory air is supplied and/or discharged via a respiratory air channel (1) in a first direction of flow (X) to a person, wherein the respiratory air flowing in the respiratory air channel (1) is exposed to UVC radiation, the method comprising the steps:
redirecting the respiratory air in all radial directions (R) to the first flow direction (X) outwards in a wider section (2) of the respiratory air channel (1), whereby the flow path and the flow cross-section in this widened section (2) of the respiratory air channel (1) increase and
irradiating the respiratory air in this widened section (2) of the respiratory air channel (1) with UVC radiation.

12. The respiratory air disinfection method according to claim 11, wherein the respiratory air is exposed to UVC radiation at the radial-outer edge in the area of the largest expansion of the flow cross-section.

13. The respiratory air disinfection method according to claim 11, wherein the respiratory air at the radial-outer edge in this widened section (2) of the respiratory air channel (1) is redirected back to the first flow direction (X).

14. The respiratory air disinfection method according to claim 11, wherein the UVC radiation is modulated in the radiation frequency.

15. The respiratory air disinfection method according to claim 11, wherein the UVC radiation is summed.
